(19) United States
(12) Patent Application Publication
(10) Pub. No.: US 2021/0338938 A1
(43) Pub. Date: Nov. 4, 2021

(54) Injection Apparatus

(71) Applicant: My Invu Limited, Broadstairs, Kent (GB)

(72) Inventors: Craig Harrison, Broadstairs, Kent (GB); Vincent May, Broadstairs, Kent (GB)

(21) Appl. No.: 17/286,568

(22) PCT Filed: Oct. 16, 2019
(86) PCT No.: PCT/EP2019/078127

§ 371 Date: Apr. 19, 2021

(30) Foreign Application Priority Data
Oct. 17, 2018 (GB) 1816928.4

(51) Int. Cl. A61M 5/315; A61M 5/20

(52) U.S. Cl. CPC A61M 5/31551; A61M 5/20; A61M 5/31528; A61M 5/31526; A61M 5/3157; A61M 2005/314; A61M 2205/8206; A61M 2205/52; A61M 2005/208; A61M 2205/35; A61M 5/31543

(57) ABSTRACT

An injection apparatus (2) comprising a housing (4) and an injector sub-assembly (6), wherein the injector sub-assembly is located within the housing; the injector sub-assembly includes a needle (20), a syringe barrel (18) and a piston (40) located within the syringe barrel; the injector sub-assembly is slidably mounted within the housing between an operative configuration in which at least a portion of the needle projects from the housing, and a retracted configuration in which the injector sub assembly is wholly located within the housing; and wherein the housing includes a housing body and needle exit door (44) pivotally connected to the body such that the door closes a needle exit aperture defined by the housing body when the injector sub-assembly is in its retracted configuration and opens when the injector sub-assembly is in its operative configuration to permit the needle to exit the housing.

The present invention relates to an injection apparatus, and in particular to an apparatus that can be used to administer discretely a dose of a medicament one or more times a day.

Multi-dose insulin injector pens are known, for example, the NovoPen 5 from Novo Nordisk. Such pens typically include a multi-dose insulin syringe barrel (known as an insulin cartridge), a needle and a mechanical mechanism for selecting and delivering a dose of insulin to the user.

Such pens look reasonably innocuous when an end cap covers the needle. However, they are unmistakably recognisable as an injection device when the cap is removed and the needle is exposed.

Manufacturers of such injector pens recommend that a new needle is fitted immediately before each injection and then removing the needle directly after an injection is administered. However, this procedure is not convenient or practical for the user, as it requires them to carry spare needles with them, which may pose a health and safety issue. It also requires the user to dispose of the used needle, which also may not be practical if the user is in a public place. Finally, the process of replacing the needle can be fiddly and inconvenient for the user, particularly if they have limited or impaired dexterity.

However, in contrast to the manufacturer's recommendations, it is understood that a needle may be used several times before it needs to be changed.

A typical insulin injection using a conventional injector pen in a public place requires the patient to follow the following steps:
1. Remove the pen cap and find somewhere safe and clean to put it, as both hands are required later;
2. Remove the inner needle cap and find somewhere safe and clean to put it;
3. Input the desired dose by rotating the dose adjuster. If the public place is poorly lit or the patient has poor or impaired eyesight (typical with many diabetics), then this step may be relatively difficult;
4. Use one hand to move clothing away from the intended injection site, whilst using the other hand to operate the injector pen;
5. Retrieve and replace the inner needle cap without sustaining injury from the exposed needle; and
6. Retrieve and replace the outer pen cap.

This procedure can be awkward and embarrassing for the user.

The present invention as claimed sets out to address or ameliorate one or more of the problems associated with conventional injector pens as discussed above.

The terms “patient” and “user” are used interchangeably herein. However, it should be appreciated that in situations in which the patient is unable to administer the required dose to themselves, the user of the device may not be the patient.

According to a first aspect of the invention, there is provided an injection apparatus comprising a housing and an injector sub-assembly, wherein the injector sub-assembly is located within the housing; the injector sub-assembly includes a needle, a syringe barrel and a piston located within the syringe barrel; the injector sub-assembly is slidably mounted within the housing between an operative configuration in which at least a portion of the needle projects from the housing, and a retracted configuration in which the injector sub assembly is wholly located within the housing; and wherein the housing includes a housing body and needle exit door pivotally connected to the housing body such that the door closes a needle exit aperture defined by the housing body when the injector sub-assembly is in its retracted configuration and opens when the injector sub-assembly is in its operative configuration to permit the needle to exit the housing.

By slidably mounting the injector sub-assembly with the housing and providing a door which is pivotally connected to the housing body, detachable covers are not required. Moreover, the interior of the housing, when the door is closed minimises or prevents potential contamination of the needle with microbes, allowing it to be re-used safely.

It will be appreciated that the needle exit door includes a fulcrum about which it pivots. The fulcrum may be located at one end of the door, in which case, the door may be said to be hingedly connected to the housing body, or the fulcrum may be located elsewhere such that the door rotates about the fulcrum relative to the housing body.

In an embodiment of the invention, the needle exit door is operatively coupled to the injector sub-assembly such that the door is moved from a closed configuration to an open configuration as the injector sub-assembly moves from its retracted configuration to its operative configuration. In this embodiment, the needle exit door automatically opens as the injector sub-assembly moves from its retracted configuration to its operative configuration and automatically closes as the injector sub-assembly is retracted and the needle is returned to the interior of the housing. Accordingly, a single action by the user may both move the injector sub-assembly and opens or closes the needle exit door as appropriate.

Suitably, the apparatus includes an assembly drive motor which drives the injector sub assembly to move between its retracted and operative configurations. Thus, the user need only operate the motor to move the injector sub-assembly from its retracted configuration to its operative configuration (or back again) and optionally also to open the needle exit door. Typically, this does not require any manual dexterity on the part of the user and can be done discretely.

The assembly drive motor may be an electric motor, in which case, the housing may include one or more batteries located therein such that the motor is powered by the or each battery. Such batteries may be rechargeable or disposable. In the case of rechargeable batteries, the apparatus may include a charging input or port, such as a suitable socket, via which the or each battery may be connected to a suitable power source to recharge the or each battery in situ. Alternatively, in the case of disposable batteries, the housing may define a battery compartment within the housing, which compartment may include a removable cover to permit access to the compartment when it is necessary to change the battery or batteries.

As most electrical motors rotate an output shaft about a longitudinal axis, the apparatus may include an assembly drive transmission disposed between the motor and the injector sub-assembly, wherein the assembly drive transmission may convert the rotary output of the motor to a linear movement of the injector sub-assembly within the housing. One example of a transmission which converts a rotational movement to a linear movement includes a screw thread and one or more threaded elements which are engaged with the screw thread. An example of such an arrangement is a captive nut threadedly coupled to the screw thread. However, the or each threaded element need not be in the form of a “nut”. As the screw thread is rotated, the threaded element(s) suitably move in a linear direction along the longitudinal axis of the screw thread. An example of how such a transmission system may be employed in an embodiment of the invention includes providing the injector sub-assembly with one or more threaded elements, providing an output shaft of the motor with an external screw thread (i.e. similar to a worm screw) and engaging the threaded element(s) carried by the injector sub-assembly with the screw thread. In such an arrangement, the motor may drive the screw thread to rotate in a first sense, which results in a linear motion of the injector sub-assembly in a first direction parallel to the longitudinal axis of the screw thread. Similarly, the motor may drive the screw thread to rotate in an opposite sense, which results in a linear motion of the injector sub-assembly in the opposite direction.

The skilled person will appreciate that the screw thread in this case defines a screw thread axis about which the helical thread is formed. This is referred to herein as the longitudinal axis of the screw thread.

In a further embodiment of the invention, the injector sub-assembly further includes a dose motor which is operatively connected to the piston, whereby the motor-driven piston is displaced axially within the syringe barrel when the dose motor is activated.

The skilled person will appreciate that the syringe barrel is typically in the form of a cylinder and defines a syringe barrel axis which is the longitudinal axis through the cylindrical syringe barrel. As such, the term “axially” means movement along the longitudinal axis of the syringe barrel.

As with the assembly drive motor discussed above, the apparatus may further include a dose transmission disposed between the dose motor and the piston which converts a rotational motion of the motor to a linear motion of the piston. Such a dose transmission may include two or more gear wheels which are meshed together and/or a screw thread/captive nut arrangement (as discussed above) and/or a worm gear arrangement.

The dose motor may be an electric motor. In such embodiments, the injection apparatus suitably includes one or more batteries located within the housing whereby the dose motor is powered by the or each battery.

In embodiments in which both the assembly drive motor and the dose motor comprise electric motors, the apparatus may include a single battery arrangement which powers both the assembly drive motor and the dose motor as appropriate, wherein the battery arrangement includes one or more battery cells.

For injection apparatus that need to be used multiple times by a user (e.g. a diabetic who needs to administer to themselves regular doses of insulin), it is useful to be able to control the dose of a medicament that is delivered by the device. Thus, the apparatus may further include a dose input component, wherein the displacement of the piston within the syringe barrel may be varied via the dose input component. For example, the dose input component may be connected to a dose controller, which in turn is connected to the piston located within the syringe barrel, such that the dose controller controls the displacement of the piston. In the context of the present invention, the term “connected” includes both directly connected and indirectly connected, where an indirect connection is defined as a connection via one or more intermediate components. The ability to vary the displacement of the piston for a specific injection event allows the user to vary the dose of the medicament that is administered by the apparatus.

The dose input component may comprise a single element (e.g. a rotary dial) or more than one elements. For example, the dose input component may include an “increase” element and a separate “decrease” element, such as “increase” and “decrease” buttons.

The assembly drive motor may be controlled by an assembly drive controller.

In a further embodiment of the invention, the apparatus includes a primary controller. The primary controller may comprise the dose controller and the assembly drive controller, or it may comprise a single controller which functions to control both the assembly drive motor and the dose motor. In other words, the dose controller and the assembly drive controller may be a single common controller. The primary controller may include a first input which is operatively connected to the dose input component and a first output which is operatively connected to the dose motor; and the primary controller converts data input via the dose input component to an output or operation signal that is sent to the dose motor. In this way, the dose input component may transmit a signal to the first input of the primary controller, which the primary controller then converts to an output signal that is sent to the motor. For example, if the user increases the dose to be delivered by the apparatus, this is input by the user via the dose input component (e.g. by rotating a dial or pressing a “dose increase” button). The primary controller receives this input and calculates a correspondingly increased operation time for the dose motor according to a pre-determined algorithm. Conversely, if the user wishes to decrease the dose, this is input by the user via the dose input component (e.g. rotating a dial in the opposite direction or pressing a “dose decrease” button), the primary controller calculates a correspondingly shorter operation time for the dose motor and corresponding signal is sent by the primary controller to the dose motor. The primary controller may be a processor that is programmed to receive inputs from the dose input component, calculate a corresponding action for the dose motor and transmit operation signals to the dose motor. The terms “controller” and “processor” may be used interchangeably herein.

In embodiments in which the apparatus includes a primary controller, the dose input component may comprise one or more buttons or a rotary dial which are electrically coupled to the input of the controller.

The primary controller may include a second input which is operatively connected to an operation input (e.g. an “activate” button). The primary controller may further include a corresponding second output, which is operatively connected to the assembly drive motor. In such an embodiment, the primary controller may activate the assembly drive motor in response to an input received from the operation input, such as a user-activated button.

Thus, the primary controller may control both the assembly drive motor and the dose motor.

In an embodiment of the invention, the apparatus includes a dose motor and a dose motor transmission, wherein the dose motor is operatively coupled to the piston located within the syringe barrel via the dose motor transmission. Thus, the dose motor transmission converts the rotary motion of the dose motor to an axial displacement of the piston within the syringe barrel. In order to increase the accuracy of the apparatus, the dose motor transmission may include a dose sensor which senses a rotation of a part of the dose motor transmission or the axial displacement of the piston. It will be appreciated that the rotation of a component of the dose motor transmission may directly correlate to the axial displacement of the piston, which in turn directly correlates to a specific output from the syringe barrel via the needle. Such an arrangement is likely to be more accurate than operating the dose motor for a predetermined period of time. Accordingly, the primary controller may calculate a specific rotation or number of rotations of the dose motor transmission and permit the dose motor to operate until the calculated rotation or number of rotations is sensed as an alternative to or in addition to calculating a specific time of operation of the dose motor.

The displacement of the piston as sensed by the dose sensor is suitably fed back to the primary controller (e.g. to a third input of the primary controller), which is able to energise the dose motor until such time that the displacement of the piston (either sensed directly or via a rotation of a component in the dose motor transmission) equates to the ejection from the syringe barrel of a desired volume of the medicament.

In addition to controlling the dose motor, the primary controller may also “count” the number of doses that have been administered by the apparatus. This count may be stored in a memory storage component which may form part of the primary controller or which may be separate to the primary controller. The primary controller may output a warning (visual and/or audible) if the selected dose to be administered exceeds the remaining volume of medicament within the syringe barrel. In such a situation, the apparatus may be prevented from ejecting a partial dose. For example, the apparatus may include a safety feature which prevents the apparatus from operating if the volume of medicament remaining in the syringe barrel is less than the desired dose. The primary controller may include data relating to the number of available doses from a new syringe barrel or it may be programmable with such data. The programming of the primary controller in such embodiments may be automatic. For example, the syringe barrel may include an RFID chip or similar device which transmits the number of doses contained within the syringe barrel. The dose count within the memory storage component may be reset automatically when a new syringe barrel is fitted to the apparatus or it may be manually reset.

The primary controller (where present) is suitably powered by one or more batteries, which may the same battery or batteries which power the assembly drive motor and/or the dose motor.

As the apparatus has the capability of causing injury if used incorrectly, the apparatus may include a safety sensor, wherein the safety sensor prevents movement of the injector sub-assembly from its retracted configuration to its operative configuration if a pre-determined condition measured by the safety sensor is not met. For example, the safety sensor may comprise a skin contact sensor which determines if the sensor is in contact with the patient's skin. Such sensors are well known and need not be described in detail herein. If the sensor senses that it is in contact with the patient's skin, then the pre-determined condition is met and the movement of the injector sub assembly from its retracted configuration to its operative configuration may be permitted. However, if the sensor senses that it is not in contact with a patient's skin, then the pre-determined condition is not met and the movement of the injector sub-assembly from its retracted configuration to its operative configuration may be prevented. In an embodiment of the invention, the safety sensor may be connected to the primary controller, such that a signal from the safety sensor which indicates its condition may be transmitted to a further input of the primary controller.

As a further safety feature, the apparatus may include a second “watchdog” controller, which prevents dosing errors in the event that the primary controller malfunctions. The second controller suitably includes inputs which are independently connected to the dose input controller, the dose sensor and/or the safety sensor. In addition, the second controller may include an output connected to the dose motor. In such an arrangement, the dose motor may be controlled such that it can only operate if it receives output signals from both the primary controller and the second “watchdog” controller. Thus, the dose motor may include a drive circuit which includes a first input connected to an output of the primary controller and a second input connected to an output of the second controller, and the drive circuit is configured to energise the drive motor only the event that output signals (i.e. operation signals) are received from both the primary and second controllers. For example, the primary and second controllers may independently check that the apparatus is operational, the pre-determined condition(s) required by the safety sensor(s) are met (e.g. the apparatus is in contact with the patient's skin), there is sufficient medicament in the syringe barrel for the selected dose, a valid dose has been selected, sufficient time has elapsed since the preceding injection event, and there is sufficient power in the batteries to complete the injection event. If both the primary and second controllers determine that the injection event can proceed, they may both, independently send an output or operation signal to the dose motor drive circuit, which then energises the dose motor. If only one or no signals are received from the primary and second controllers, the dose motor drive circuit will not energise the drive motor and an error signal may be emitted.

It is often necessary for a patient to know when they last used the injection apparatus and how much of the medicament they injected. As such, the apparatus may further include a memory which stores data relating to previous uses of the apparatus. For example, the data may include the time since or when the apparatus was last used and/or the dose dispensed by the apparatus. Accordingly, the memory may include a counter which measures the time since a preceding event or a clock which records the time when preceding events occurred. In addition, as noted above, the memory may also store a cumulative count of the number of doses that have been administered from the syringe barrel located within the apparatus.

The apparatus suitably also includes a display screen carried by the housing. The display screen may display, for example, the desired dose to be administered, the elapsed time since the previous injection event or the time at which the previous injection event occurred, and/or the dose that was administered at the previous injection event.

As a further safety feature, the apparatus may further include an operation lock which has a locked configuration in which movement of the injector sub-assembly from its retracted configuration to it operative configuration is prevented and an unlocked configuration in which the movement of the injector sub-assembly from its retracted configuration to its operative configuration is permitted. Such an operation lock may be in the form of a mechanical lock which physically prevents movement of the injector sub-assembly. Additionally or alternatively, the operation lock may be an electronic lock which prevents power being supplied to the assembly drive motor and/or the dose motor (where present) in its locked configuration.

For example, the operation lock may comprise a power button which electrically connects the electrical components of the apparatus (e.g. the primary controller, the assembly drive motor and/or the dose motor) to the electrical power source when switched on (i.e. in its unlocked configuration) and which isolates the electrical components of the apparatus from the electrical power source when switched off (i.e. in its locked configuration).

As such, in certain embodiments of the invention, the apparatus will only function if the operation lock is in its unlocked configuration and the safety sensor senses that one or more pre-determined conditions are met.

In an embodiment, the apparatus includes a biasing element or a control element which returns the injector sub-assembly to its retracted configuration after each use or after a pre-determined period of time. In this way, the injector sub-assembly may automatically be configured in its retracted configuration after use. This avoids the need for the user to take any further action once the medicament has been injected.

Suitably, in embodiments in which the apparatus includes an operation lock as discussed above, the biasing element or the control element may also configure the operation lock in its locked configuration when it returns the injector sub-assembly to its retracted configuration. As such, the operation lock then needs to be disengaged (i.e. configured in its unlocked configuration) before the apparatus can be used again.

The injector sub-assembly of the apparatus may be configured to utilise known syringe barrels and needles. Such known syringe barrels are typically filled with a pre-determined number of doses of the medicament. However, it is useful for the user to know how many doses remain in the syringe barrel. As such, the housing body may define a window through which the syringe barrel of the injector sub-assembly is visible.

It is well known that injections can be painful. This is because large numbers of small nerve fibres are located within and beneath the skin which transmit pain signals to the brain if they are stimulated. However, according to Gate Control Theory, pressure and vibration at an injection site can interfere with the ability of the small nerve fibres to transmit the pain signals to the brain. In this case, it is believed that the pressure and vibration at the injection site stimulates larger nerve fibres at the injection site, which can reduce or inhibit the flow of “pain” signals transmitted by the small nerve fibres by closing the gate to the signals from the small nerve fibres.

In view of Gate Control Theory, the apparatus may include a plurality of pins located adjacent to the needle exit aperture. The apparatus may further include a vibrator arranged to vibrate the pins and a vibrator controller which controls the vibrator. According to this arrangement, the pins may exert pressure and vibration to the skin surrounding the injection site. The vibrator controller may be connected to or form a part of the primary controller. For example, just prior to an operation signal being sent by the primary controller to the assembly drive motor, an operation signal may be sent to the vibrator, so that the pins are vibrated and the skin area adjacent to the pins is de-sensitised prior to the needle exiting the needle exit aperture.

In an embodiment of the invention, the pins may define a ring or annulus around the needle exit aperture.

In the field of diabetes management, it is known to implant a glucose sensor beneath the skin of a patient. Such sensors typically include near field transmitters which transmit a signal when the sensor is energised by a nearby power source. The subject of near field communication is relatively well understood and need not be described in detail herein. In order to communicate with such implanted sensors, the apparatus of the invention may further include a near field communication (NFC) transceiver, which is capable of powering the sensor when the apparatus is located adjacent to the sensor and receiving data from it, for example relating to blood glucose levels. The data from the NFC transceiver may be received by the primary controller and a suitable dose based on the data may be calculated by the primary controller. The calculated dose may be accepted by the user or it may be altered using the dose input component.

The NFC transceiver may also be capable of transmitting data from the primary controller and/or a memory component to a data receiver, for example a mobile device running appropriate software or a computer. Such data may be useful in managing the use of the apparatus and/or the medical condition which is being managed by the apparatus.

The skilled person will appreciate that the features described and defined in connection with the aspect of the invention and the embodiments thereof may be combined in any combination, regardless of whether the specific combination is expressly mentioned herein. Thus, all such combinations are considered to be made available to the skilled person.

An embodiment of the invention will now be described, by way of example only, with reference to the accompanying drawings in which:

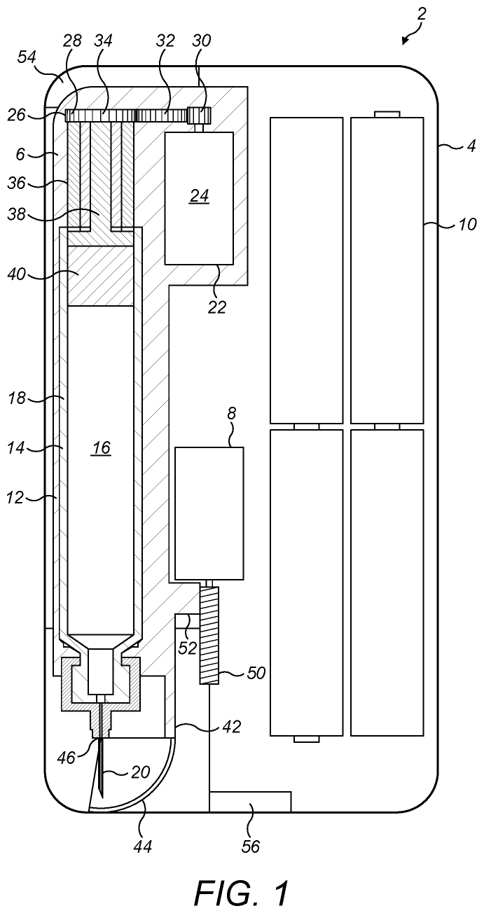

FIG. 1 shows a cross-section through an injection apparatus according to the invention in its retracted configuration;

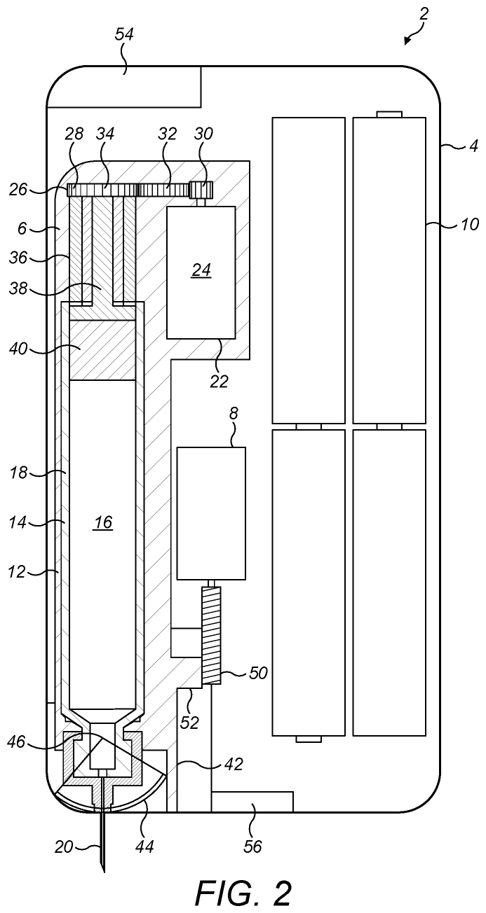

FIG. 2 shows a cross-section through the injection apparatus shown in FIG. 1 when in its operative configuration;

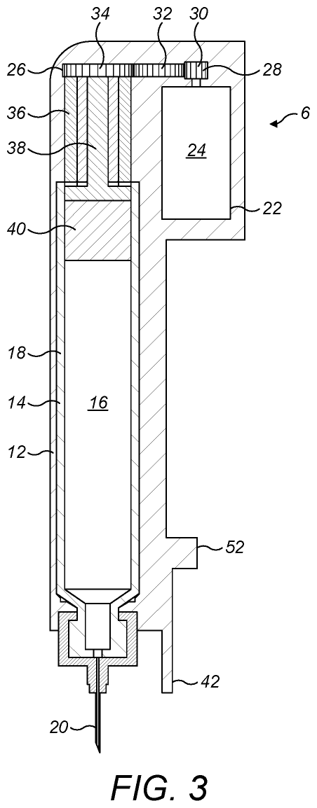

FIG. 3 shows a cross-section through the injector sub-assembly which forms a part of the invention as shown in FIGS. 1 and 2;

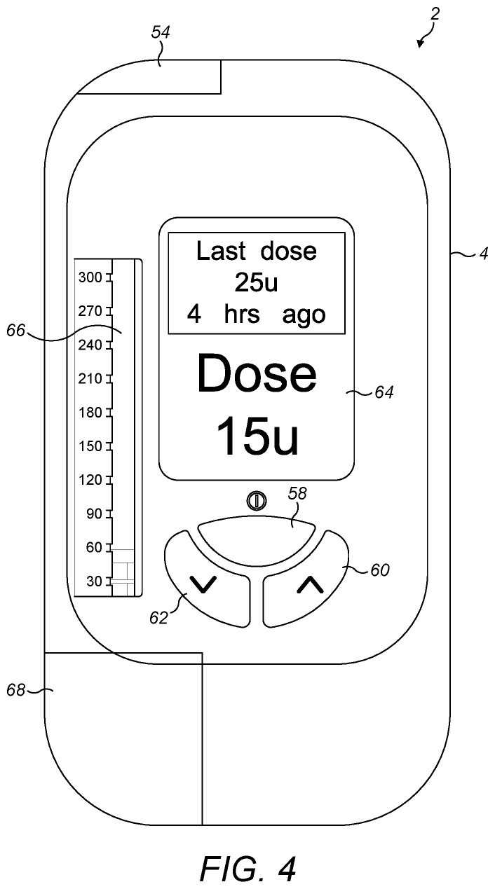

FIG. 4 shows a front elevational view of the injection apparatus shown in FIG. 1; and

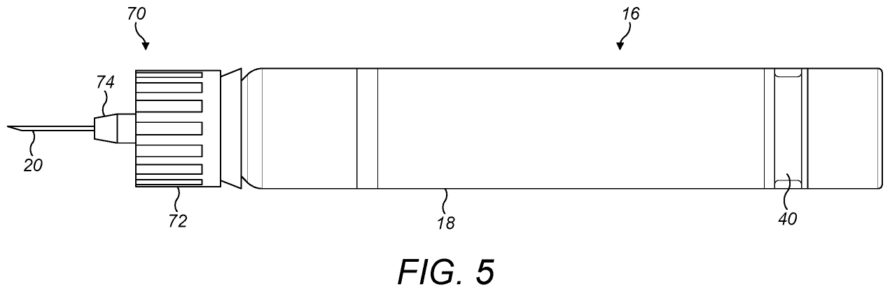

FIG. 5 shows a refill cartridge for use with the injection apparatus.

For the avoidance of doubt, the skilled person will appreciate that in this specification, the terms “up”, “down”, “front”, “rear”, “upper”, “lower”, “width”, etc. refer to the orientation of the components as found in the example when installed for normal use as shown in the Figures.

FIGS. 1 and 2 show a cross-section through an injection apparatus 2 according to the invention. The apparatus 2 comprises a housing body 4 within which is located an injector sub-assembly 6 (shown by itself in FIG. 3), an assembly drive motor 8 and four “AAA” size batteries 10.

The injector sub-assembly 6 includes a body 12 which defines a first recess 14 that is shaped and configured to receive therein a conventional insulin cartridge 16 (shown in FIG. 5) which includes a syringe barrel 18 and a needle 20. The body 12 also defines a second recess 22, within which is located a dose motor 24, and a third recess 26 within which is located a dose transmission 28.

The dose motor 24 is powered by the batteries 10 and is controlled by a primary processor (not shown). The primary processor is a conventional processor which energises the motor 24 for a period of time that is sufficient to deliver the desired dose of a medicament stored within the syringe barrel 18.

The dose transmission includes a first spur gear 30, an intermediate spur gear 32 and a driven spur gear 34, wherein the first spur gear 30 is driven by the dose motor 24 and is meshed with the intermediate spur gear 32, and the intermediate spur gear 32 is meshed with the driven spur gear 34 such that rotation of the first spur gear 30 by the dose motor 24 is transmitted to the driven spur gear 34 via the intermediate spur gear 32. The driven spur gear 34 drives a conventional telescopic screw jack 36 which in turn drives a piston contact end 38 of the screw jack 36 in a linear direction, which is up and down as shown in FIGS. 1 and 2.

The transmission further includes a rotation sensor (not shown) which senses the rotation of the driven spur gear 34. The rotation of the driven spur gear equates to a corresponding linear motion of the piston contact end 38 of the screw jack 36, which in turn corresponds to a swept volume of the syringe barrel 18. The rotation sensor feeds back to the primary processor which controls the dose motor 24 such that the desired dose is ejected from the syringe.

The piston contact end 38 of the telescopic screw jack 36 engages a piston 40 disposed within the syringe barrel 18, the piston 40 forming part of the conventional insulin cartridge 16.

The injector sub-assembly 6 further includes a downwardly projecting leg 42 which is operatively connected to a needle exit door 44. The needle exit door is pivotally connected to the housing body 4 such that it is able to pivot about a fulcrum 46. As the injector sub-assembly 6 moves downwards, it causes the needle exit door 44 to pivot about the fulcrum 46. The needle exit door 44 defines therein an opening (not shown) which aligns with the needle 20 as the injector sub-assembly 6 moves downwards and the needle exit door 44 pivots about the fulcrum 46. However, when the injector sub-assembly 6 is in its fully retracted configuration, as shown in FIG. 1, the opening in needle exit door 44 is out of alignment with the needle 20 such that the needle 20 is covered by the needle exit door 44 and the interior of the housing 4 is essentially closed against the ingress of foreign particulate matter.

The injector sub-assembly 6 is able to move linearly within the housing 4 (in an up and down direction as shown in FIGS. 1 and 2). This linear movement is driven and controlled by the assembly drive motor 8.

A shaft 50 which defines an outer helical thread is secured to the output shaft of the assembly drive motor 8. Corresponding threaded elements (not shown) are provided on a distal end of an arm 52 which projects from the injector sub-assembly 6, such that the threaded elements of the arm 52 mesh with the outer helical thread of the shaft 50. In this way, the threaded elements carried by the arm 52 and the threaded shaft 50 together form a pseudo “rack and pinion” type transmission arrangement, which converts the rotational motion of the shaft 50 when driven by the assembly drive motor 8 to a linear motion of the injector sub-assembly 6. The skilled person will appreciate that instead of the “open” threaded elements, the arm 52 may carry at its distal end a captive nut through which the shaft 50 passes.

The apparatus 2 further includes a secondary processor (not shown) and an assembly drive motor control circuit (also not shown). The primary processor and the secondary processor are both connected to the drive motor control circuit and the drive motor control circuit will only energise the assembly drive motor 8 if positive control signals are received from both the primary and secondary processors. A similar arrangement is provided for the dose motor 24. Accordingly, the secondary processor acts as a failsafe or watchdog for the primary processor to ensure the safe operation of the apparatus 2.

At the top of the housing body 4 is an “inject” button 54 which activates the dose motor 24 if the processor has determined that all relevant safety conditions have been met.

One of the safety conditions is sensed by a skin contact sensor 56 located at the bottom of the housing body 4. The skin contact sensor 56 comprises a capacitive sensor element which is configured to sense contact with skin. The skin contact sensor is electrically connected to an input of the processor, and the processor is programmed not to permit operation of the assembly drive motor 8 or the dose motor 24 unless the skin contact sensor 56 is in contact with a portion of skin 1 of the patient.

FIG. 3 shows the injector sub-assembly 6 by itself.

FIG. 4 shows a front view of the apparatus 2. On the front of the housing body 4 is provided an on/off button 58, a dose increase button 60 and dose decrease button 62. In order to activate the apparatus 2, the patient must first press the on/off button 58. Thus, the on/off button 58 forms an operation lock for the apparatus 2. The patient then selects the desired dose of the medicament (e.g. insulin) using either the dose increase button 60 or the dose decrease button 62. The selected dose is displayed on a display screen 64 located on the front of the housing body 4. The display screen 64 also displays the last dose that was administered by the apparatus 2 and the elapsed time since the last dose.

So that the patient has a visible indication of the number of doses remaining in the syringe barrel 18, an elongate window 66 is provided on the front face of the housing body 4 such that the window 66 overlies the syringe barrel 18 located within the housing body 4.

It is necessary periodically to replace the insulin cartridge 16, or the needle 20. Accordingly, a hinged cover 68 is provided over a portion of the first recess 14 which permits access to the recess 14 and the insulin cartridge 16 located therein. When the hinged cover 68 is rotated away from the housing body 4, the patient is able to access the needle 20 carried by the cartridge 16 or, if desired, to remove the cartridge 16 in its entirety from the first recess 14.

The apparatus 2 shown in FIG. 4 further includes a near field communications transceiver 70, which is powered by the batteries 10 and which is able to communicate with devices implanted under the skin of a patient, such as a continuous glucose monitoring implant, for example the Freestyle Libre™. The NFC transceiver may also communicate with mobile devices to transfer operational data (such a historical data relating to amounts of medicament injected and when the injection events occurred) for patient records.

The apparatus 2 further includes a ring of gate control pins 72 located around the needle exit aperture (i.e. the aperture defined through the housing 4, through which the needle 20 passes when in use). The gate control pins 72 are connected to a vibrator (not shown), which in turn is controlled by the primary processor, such that the pins apply a vibrating force to the patient's skin adjacent to the injection site immediately prior to the needle 20 exiting the housing. As noted above, this decreases the pain signals associated with the injection event.

The insulin cartridge 16 is shown in more detail in FIG. 5. Suitable cartridges 16 are commercially available, for example, the Penfill 3 mL insulin cartridge from Novo Nordisk. The cartridge 16 comprises a syringe barrel 18 and a needle 20. The needle 20 forms part of a needle assembly 80 which comprises a threaded connector portion 82, a needle boss 84 which extends from the threaded connector portion 82 and the needle 20 which is secured to the needle boss 84.

The threaded connector portion 82 threadedly engages a corresponding thread formed a distal end of the syringe barrel 18 in order to secure the needle 20 to the syringe barrel 18. The syringe barrel also includes the piston 40 which forms a liquid-tight seal with an interior surface of the syringe barrel 18. The proximal end of the syringe barrel 18 is open in use to permit access to the piston 40 by the piston contact end 38 of the telescopic screw jack 36.

When a new cartridge 16 is inserted into the apparatus 2, the apparatus enters an “airshot” mode, which permits the dose motor 24 to operate for a short period of time without the apparatus 2 being in contact with the skin or the needle being expose in order to purge any air from the syringe barrel 18.

In use, a patient removes the operation lock by pressing and holding the on/off button 58 until the device powers-up. The patient then enters the desired dose of the medicament (e.g. insulin) using the dose increase button 60 or the dose decrease button 62. The selected dose is displayed on the display screen 64. The patient then exposes a portion of skin at the desired injection site, contacts the skin with the bottom of the housing body 4 and presses the inject button 54.

The skin contact sensor 56 senses that the housing body 4 is in contact with a portion of skin and sends a signal to the primary and secondary processors to confirm that this necessary condition for safe operation of the apparatus 2 is met. The processors then send a positive control signal to the drive motor control circuit and the assembly drive motor 8 is energised, which in turn rotates the threaded shaft 50. The engagement between the threaded shaft 50 and the threaded elements carried by the arm 52 of the injector sub-assembly 6 cause the injector sub assembly to move linearly downwards towards the bottom of the housing body 4. The downward movement of the injector sub-assembly also causes the needle exit door 44 to open as a result of the connection between the leg 42 of the injector sub-assembly 6 and the needle exit door 44, and the pivotal coupling of the needle exit door 44 about the fulcrum 46. The opening of the needle exit door 44 by the downward movement of the injector sub-assembly 6 permits the needle 20 to project through the needle outlet aperture, beyond the bottom of the housing body 4 and through the skin 1 of the patient.

When the injector sub-assembly 6 is in its operative configuration (i.e. the injector sub-assembly has reached the end of its downward travel), the drive motor control circuit then interrupts the power to the assembly drive motor 8. The primary and secondary processors then send a control signal to the dose motor 24 which is energised and drives the piston 40 disposed within the syringe barrel 18 downwards by a predetermined distance via the dose transmission 28. The processors calculates the volume of the medicament to be dispensed based on the desired dose of the medicament. It then calculates the required displacement of the piston to dispense the calculated volume of the medicament, and finally it calculates the corresponding rotation of the driven spur gear 34 required to displace the piston by the desired distance. The primary and secondary processors control the dose motor 24 such that it is energised until the driven spur gear 34 has completed the calculated rotation or rotations, as sensed by the dose sensor, after which the dose motor 24 is disconnected from the electrical power source provided by the batteries 10.

In an alternative embodiment (not shown), the apparatus 2 includes a dose sensor which either directly senses displacement of the piston 40 or which indirectly senses displacement of the piston 40 via the rotation of a different component of the dose transmission 28. The processor is able to determine the volume of the medicament that has been ejected from the apparatus based on the displacement of the piston 40, as sensed by the dose sensor. The processor controls the power supplied to the dose motor 24 in response to the displacement of the piston as sensed by the dose sensor.

After the power to the dose motor 24 has been interrupted, the primary and secondary processors control the assembly drive motor 8 to rotate in the opposite sense to the previous operation such that the injector sub-assembly 6 is driven upwards. This upward movement of the injector sub-assembly 6 retracts the needle 20 from the skin 1 of the patient and closes the needle exit door 44.

Once the injector sub assembly 6 is in its retracted configuration, the operative lock is re-engaged whereby the apparatus 2 can only be re-activated when the user presses and holds the on/off button 58 once again. The data displayed on the display screen in connection with the previous dose and the time since the previous dose was administered is then updated.

It will be appreciated that the only actions required by the user in order to effect the administration of the medicament within the syringe barrel 18 are to switch the device on, select the desired dose, hold the device against a portion of skin and press the inject button 54.

1. An injection apparatus comprising a housing and an injector sub-assembly, wherein the injector sub-assembly is located within the housing; the injector sub-assembly includes a needle, a syringe barrel and a piston located within the syringe barrel; the injector sub-assembly is slidably mounted within the housing between an operative configuration in which at least a portion of the needle projects from the housing, and a retracted configuration in which the injector sub assembly is wholly located within the housing; and wherein the housing includes a housing body and needle exit door pivotally connected to the housing body such that the door closes a needle exit aperture defined by the housing body when the injector sub-assembly is in its retracted configuration and opens when the injector sub-assembly is in its operative configuration to permit the needle to exit the housing.

2. An injection apparatus according to claim 1, wherein the needle exit door is operatively coupled to the injector sub-assembly such that the door is moved from a closed configuration to an open configuration as the injector sub-assembly moves from its retracted configuration to its operative configuration.

3. An injection apparatus according to claim 1, wherein the apparatus further includes an assembly drive motor which drives the injector sub assembly to move between its retracted and operative configurations.

4. An injection apparatus according to claim 3, wherein the drive motor is an electric drive motor and the injection apparatus further includes one or more batteries located within the housing, wherein the drive motor is powered by the or each battery.

5. An injection apparatus according to claim 3, wherein the injection apparatus further includes a drive transmission disposed between the drive motor and the injector sub-assembly.

6. An injection apparatus according to claim 5, wherein the drive transmission includes an external screw thread and one or more threaded elements which engage with the external screw thread.

7. An injection apparatus according to claim 6, wherein the or each threaded element is carried by the injector sub-assembly and the screw thread is carried externally by an output shaft from the motor, whereby rotation of the screw thread by the motor causes the injector sub-assembly to move linearly within the housing.

8. An injection apparatus according to claim 1, wherein the injector sub-assembly further includes a dose motor which is operatively connected to the piston located within the syringe barrel, whereby the piston is urged to move axially within the syringe barrel when the dose motor rotates.

9. An injection apparatus according to claim 8, wherein the injector sub-assembly further includes a dose transmission disposed between the dose motor and the piston, wherein the piston is driven by the motor via the dose transmission.

10. An injection apparatus according to claim 8, wherein the dose motor is an electric motor; the injection apparatus includes one or more batteries located within the housing; and the dose motor is powered by the or each battery.

11. An injection apparatus according to claim 8, wherein the apparatus further includes a dose input component, wherein the displacement of the piston within the syringe barrel is determined by the dose input component.

12. An injection apparatus according to claim 11, wherein the apparatus includes a controller, wherein the controller includes an input which is operatively connected to the dose input component; the controller includes an output which is operatively connected to the dose motor; and the controller converts data input via the dose input component to an output signal which controls the operation of the dose motor.

13. An injection apparatus according to claim 1, wherein the apparatus includes a safety sensor, wherein the safety sensor prevents movement of the injector sub-assembly from its retracted configuration to its operative configuration if a pre-determined condition measured by the sensor is not met.

14. An injection apparatus according to claim 13, wherein the safety sensor comprises a skin contact sensor and the pre-determined condition is contact of the sensor with the skin of a user.

15. An injection apparatus according to claim 1, wherein the apparatus includes a memory which stores data relating to previous uses of the apparatus.

16. An injection apparatus according to claim 15, wherein the data includes the time since the previous use of the apparatus and/or the previous dose dispensed by the apparatus.

17. An injection apparatus according to claim 15, wherein the syringe barrel contains a pre-determined number of doses of a liquid pharmaceutical composition and the memory further includes data relating to the number of doses of the composition remaining in the syringe barrel.

18. An injection apparatus according to claim 1, wherein the apparatus further includes an operation lock which has a locked configuration in which movement of the injector sub-assembly from its retracted configuration to it operative configuration is prevented and an unlocked configuration in which the movement of the injector sub-assembly from its retracted configuration to its operative configuration is permitted.

19. An injection apparatus according to claim 1, wherein the apparatus includes a biasing element or a control element which returns the injector sub-assembly to its retracted configuration after each use or after a pre-determined period of time.

20. An injection apparatus according to claim 19, wherein the apparatus further includes an operation lock which has a locked configuration in which movement of the injector sub-assembly from its retracted configuration to it operative configuration is prevented and an unlocked configuration in which the movement of the injector sub-assembly from its retracted configuration to its operative configuration is permitted; and wherein the biasing element or the control element configures the operation lock in its locked configuration when it returns the injector sub-assembly to its retracted configuration.

21. An injection apparatus according to claim 1, wherein the housing body defines a window through which the syringe barrel is visible.

22. An injection apparatus according to claim 1, wherein the apparatus further includes a plurality of pins located adjacent to the needle exit aperture, wherein the apparatus further includes a vibrator which vibrates the pins when activated.

23. An injection apparatus according to claim 1, wherein the apparatus further includes a near field communication transceiver.
